(19) United States
(12) Patent Application Publication
(10) Pub. No.: US 2005/0025771 A1
(43) Pub. Date: Feb. 3, 2005

(54) Antitumor agents comprising a targeting portion and an immune response triggering portion

(75) Inventors: Thomas E. Wagner, Greenville, SC (US); Yanzhang Wei, Greer, SC (US)

Correspondence Address: FOLEY AND LARDNER;SUITE 500, 3000 K STREET NW, WASHINGTON, DC, 20007, US

(73) Assignee: Greenville Hospital System

(21) Appl. No.: 10/792,390

(22) Filed: Mar. 4, 2004

Related U.S. Application Data
(60) Provisional application No. 60/451,253, filed on Mar. 4, 2003.

(51) Int. Cl. A61K 39/395

(52) U.S. Cl. 424/155.1; 424/178.1

(57) ABSTRACT

The present invention provides an antitumor agent comprising a targeting portion and an immune response triggering portion. The targeting portion may be an antibody fragment or a tumor vasculature binding peptide. The immune response triggering portion may be an Fc fragment of immunoglobulin G (IgG), a fragment of the Fc fragment of IgG that exhibits the same biological function as the Fc region, or the extracellular domain of foreign major histocompatability complex (MHC). The antitumor agent is useful for inhibiting tumor growth, inhibiting tumor angiogenesis and treating diseases associated with neovascularization.

CROSS-REFERENCE TO RELATED APPLICATION

This application claims the benefit of U.S. Provisional Application No. 60/451,253 filed Mar. 4, 2003, which is incorporated herein by reference in its entirety.

### BACKGROUND OF THE INVENTION

Present methods for tumor treatment, especially cancer treatment, remain sub-optimal because they often fail to destroy or remove all of the tumor or cancer cells. Furthermore, present methods are accompanied by severe complications and adverse side effects due to a lack of specificity in targeting cells for destruction or removal.

A key to the development of successful antitumor agents is the ability to design agents that will selectively kill tumor cells, while having relatively little, if any, effect upon normal tissues. This goal has been difficult to achieve because there are few qualitative differences between neoplastic and normal tissues. To accomplish this goal, researches have focused on identifying tumor-specific “marker antigens” that can serve as immunological targets both for chemotherapy and diagnosis. Many tumor specific, or quasi-tumor-specific (“tumor-associated”), markers have been identified as tumor cell antigens that can be recognized by specific antibodies. It is generally the case that tumor specific antibodies will not in and of themselves exert sufficient antitumor effects to make them useful in cancer therapy. Therefore, tumor specific antibodies may be labeled with a cytotoxic agent that is designed to destroy tumor cells.

Another targeting approach is vascular specific targeting, wherein the tumor vasculature is targeted rather than the tumor itself. The tumor vasculature has become a prime target for cancer therapy because of its essential role in the growth and survival of all solid tumors (Folkman, J. 1997 Cancer: Principles and Practice of Oncology. Lippincott-Raven, New York. pp. 3075-3087). This is because new blood vessel growth (angiogenesis) is required for continued tumor growth (Risau, W. 1997. Mechanisms of angiogenesis. Nature 386: 671-674; Zetter, B. R. 1998. Angiogenesis and tumor metastasis. Annu. Rev. Med. 49: 407-424; and Bicknell, R. 1997. Tumor Angiogenesis. Oxford University Press, Oxford. pp. 19-28). For example, the αvβ3 integrins have been found to be tumor specific, and therefore, excellent tumor targets (Brooks, P. C., Clark, R. A., and Cheresh, D. A. 1994. Requirement of vascular integrin αvβ3 for angiogenesis. Science 264:569-571). An αvβ3 integrin specific antibody has been developed (Tarli, L., Balza, E., Viti, F., Borsi, L., Castellani, P., Berndorff, D., Dinkelborg, L., Neri, D., and Zardi, L. 1999. A high-affinity human antibody that targets tumoral blood vessels. Blood 94: 192-198; Marty, C., Scheidegger, P., Ballmer-Hofer, K., Klemenz, R., and Schwendener, R. A. 2001. Production of functionalized single-chain Fv antibody fragments binding to the ED-B domain of the B-isoform of fibronectin in Pichia pastoris. Protein Expression and Purification 21: 156-164) and used to target tumors with immunotoxins (Carnemolla, B., Borsi, L., Balza, E., Castellani, P., Meazza, R., Berndt, A., Ferrini, S., Kosmehl, H., Neri, D., and Zardi, L. 2002. Enhancement of the antitumor properties of interleukin-2 by its targeted delivery to the tumor blood vessel extracellular matrix. Blood 99: 1659-1665). On the other hand, bacteria phage display technology has been used to identify a group of peptides containing the Arg-Gly-Asp (RGD) motif, which have high affinity for αvβ3 integrins and to home to tumors (Pasqualini, R.,Koivunen, E., and Ruoslahti, E. 1997. αv integrins as receptors for tumor targeting by circulating ligands. Nature Biotech. 15:542-546; Assa-Munt, N., Jia, X., Laakkonen, P., and Ruoslahti, E. 2001. Solution structures and integrin binding activities of an RGD peptide with two isomers. Biochemistry 40: 2373-2378). These peptides have the potential to target tumor neo-vasculature with toxins such as pro-apoptotic peptides (Ellerby, H. M., Arap, W., Ellerby, L. M., Kain, R., Andrusiak, R., DelRio, G., Krajewski, S., Lombardo, C. R., Rao, R., Ruoslahti, E., Bredesen, D. E., and Pasqualini, R. 1999. Anti-cancer activity of targeted pro-apoptotic peptides. Nature Med. 5: 1032-1038).

There is a need for tumor therapies having little or no adverse side effects and also having great specificity and thoroughness in the destruction and removal of tumor cells.

### SUMMARY OF THE INVENTION

The present invention provides an antitumor agent comprising a targeting portion (TP) and an immune response triggering portion (IRTP). The TP may be an antibody fragment or a tumor vasculature binding peptide. In some embodiments, the antibody fragment is a single chain antibody. In other embodiments, the tumor vasculature binding peptide comprises arginine-glycine-aspartate (RGD), asparagine-glycine-arginine(NGR), or glycine-serine-leucine (GSL). The IRTP may be an Fc fragment of immunoglobulin G (IgG), a fragment of the Fc fragment of IgG that exhibits the same biological function as the Fc region, or the extracellular domain of foreign major histocompatability complex (MHC). In some embodiments, the antitumor agent is used to inhibit tumor growth. In other embodiments, the antitumor agent is used to inhibit tumor angiogenesis. In some embodiments, the antitumor agent is used for treating a disease associated with neovascularization, such as cancer.

In addition to the gene therapy methods, contemplated herein are methods for producing RGD/mFc fusion proteins in large scale using bacterium fermentation technology as another option.

### BRIEF DESCRIPTION OF THE DRAWINGS

FIG. 1. Diagram of the RGD/mFc fusion protein. RGD=peptide ACDCRGDCFCG; H=hinge region of mouse IgG; CH2 and CH3=constant regions of the Fc domain of mouse IgG.

FIGS. 2A-B. Detection of RGD/mFc expression. (A) FACS analysis of regular B16F0 cells and RGD/mFc expressing B16F0 cells (B16F0/RGD/mFc), (B) Western blot analysis of supernatant. 1, positive control from kit; 2, supernatant from regular B16F0 cell culture; 3, supernatant from RGD/mFc B16F0 cells.

FIGS. 3A-F. Fluorescent microscopy analysis of Matrigel Implants. (A) Matrigel implant containing no B16F0 cells, (B) 20 μm frozen sections of Matrigel implant containing no B16F0 cells, (C) Matrigel implant containing regular B16F0 cells, (D) 20 μm frozen sections of Matrigel implant containing regular B16F0 cells, (E) Matrigel implant containing RGD/mFc expressing B16F0 cells, (F) 20 μm frozen sections of Matrigel implant containing RGD/mFc expressing B16F0 cells.

FIG. 4. Immunohistochemical analysis of Matrigel implants. (A) Matrigel with no B16F0 cells, (B) Matrigel implant containing B16F0 cells, (C) Matrigel implant containing RGD/mFc expressing B16F0 cells.

FIG. 5. Immunohistochemical analysis of tumors. (A) Representative tumors of RGD/mFc expressing B16F0 cells (left mouse) and regular B16F0 cells (right mouse); (B) Tumor from mouse injected with regular B16F0 cells; (C) Tumor from mouse injected with RGD/mFc expressing B16F0 cells; (D) tumor microvascular density.

FIG. 6. Tumor growth study. Squares=tumor from mouse injected with regular B16F0 cells; Diamonds=tumor from mouse injected with RGD/mFc expressing B16F0 cells; ***=P<0.001.

FIG. 7. Survival study. 2×10⁵ B16F0 cells or RGD/mFc expressing B16F0 cells were subcutaneously injected into C57BL/6J mice (8 mice/group). When the tumor size reaches 20 mm in diameter, the mouse is considered dead.

FIG. 8. Nucleic acid sequence encoding the extracellular domain of H-2K^{d} (nucleotides 1-912), the hinge region (nucleotides 912-975), the variable region of anti-PSMA heavy chain (nucleotides 976-1326), a linker sequence (nucleotides 1327-1410), and the variable region of anti-PSMA light chain (nucleotides 1411-1749).

FIGS. 9A-B. (A) Diagram of the H2K^{d}/scPSMA fusion protein. scFv=variable regions of light and heavy chains of anti-human PSMA as a single chain; MHC=extracellular domain of mouse H-2K^{d} (B) RT-PCR analysis of clones of B16F0 cells transfected with plasmid pH2-K^{d}/PSMA_{VH+VL}. Lane 1: Marker; lane 2: Clone #1; lane 3: Clone #2; lane 4: Clone #3; lane 5: Clone #7; lane 6: B16F0.

FIG. 10A-D. FACS analysis of H2K^{d}/scPSMA expression in stable cell lines of B16F0 transfected with pH2-K^{d}/PSMA_{VH+VL} plasmid. (A) cell line # 3 cells were stained with FITC conjugated anti-H-2K^{d} antibody, (B) cell line # 3 cells were stained with FITC conjugated anti-mouse IgG antibody, (C) cell line # 7 cells were stained with FITC conjugated anti-H-2K^{d} antibody, (D) cell line # 7 cells were stained with FITC conjugated anti-mouse IgG antibody. The dotted lines are isotype controls.

FIG. 11A-B. Pulmonary metastasis analysis. (A) Lung tumor nodules, (B) Total tumor weight (***: P<0.001). Group 1: B16F0 cells; group 2: B16F0/PSMA cells; group 3: B16F0/H2K^{d}/scPSMA cells; group 4: B16F0/PSMA/H2K^{d}/scPSMA cells, clone #3; group 5: B16F0/PSMA/H2K^{d}/scPSMA cells, clone #7.

FIG. 12. Survival study. Diamond=B16F0 cells; Square=B16F0/PSMA cells; Circle=B16F0/H2K^{d}/scPSMA cells; Cross Squre=B16F0/PSMA/H2K^{d}/scPSMA#3 cells; and Triangel=B16F0/PSMA/H2K^{d}/scPSMA#7 cells.

FIG. 13. Tumor growth study. Diamond=B16F0 cells; Square=B16F0/PSMA cells; Circle=B16F0/H2K^{d}/scPSMA cells; Triangle=B16F0/PSMA/H2K^{d}/scPSMA#3 cells; and Cross Square=B16F0/PSMA/H2K^{d}/scPSMA#7 cells.

FIG. 14. Tumor growth study for human prostate cells. Square=remote tumor; Diamond=injected tumor; and Circle=control tumor. Vertical arrows indicate time at which virus particles were directly injected into tumors.

FIG. 15. Tumor growth study for TC-1 cells. Square=remote tumor; Diamond=injected tumor; Circle=control tumor (LacZ); and Triangle=control tumor (PBS). Vertical arrows indicate time at which virus particles were directly injected into tumors.

FIG. 16. Anti-tumor growth of RGD/mFc delivered through adenovirus in DU-145 human prostate tumor cells. 5×10⁶ DU-145 tumor cells were intradermally injected into each of three groups of nude mice (5 mice/group) in both flanks. Starting at day 21, adenoviral particles encoding RGD/mFc or LacZ (10⁸ PFU/50 μl PBS) were injected into the tumors in the right flank. The injection was repeated four times at a three-day interval. Tumors were measured starting at day 21 and the average tumor volumes are presented. Open circle: adenoviral particles encoding Lac Z injected tumors; open diamond: adenoviral particles encoding RGD/mFc injected tumors; open square: tumors on the left flank of the mice whose tumors on right flank received adenoviral particles encoding RGD/mFC. Arrows indicate the vector injections.

FIG. 17. Anti-tumor growth of RGD/mFc delivered through adenovirus in TC-1 cells. 2×10⁵ TC-1 tumor cells were intradermally injected into each of three groups of C57BL/6J mice (8 mice/group) in both flanks. Starting at day 10, adenoviral particles encoding RGD/mFc (10⁸ PFU/50 μl PBS) were injected into the tumors in the right flank. In one control group, the same amount of adenoviral particles encoding Lac Z were injected into tumors in the right flank; in another control group, 1XPBS was injected. The injection was repeated four times at three-day intervals. Tumors were measured starting at day ten and the average tumor volumes are presented. Open triangle: PBS injected tumors; open circle: adenoviral particles encoding Lac Z injected tumors; open diamond: adenoviral particles encoding RGD/mFc injected tumors; open square: tumors on the left flank of the mice whose tumors on right flank received adenoviral particles encoding RGD/mFC. Arrows indicate the vector injections.

### DETAILED DESCRIPTION

The present invention provides compositions and methods for selectively destroying a target cell, like a cancer cell. The composition is an antitumor agent that comprises a TP linked to an IRTP. The TP binds specifically to the target cell, while the IRTP triggers an immune response that causes the cell to be destroyed. Methods of making and using the antitumor agent are also described.

The Targeting Portion (TP)

The TP may be any moiety that is capable of selectively binding to a target cell. For example, the TP may be an antibody fragment or a tumor vasculature binding peptide.

In some embodiments, the TP is an antibody fragment that selectively binds to a target cell. Antibody fragments useful in the present invention are F(ab′)₂, F(ab)₂, Fab′, Fab, Fv and the like, including hybrid fragments. Also useful are any subfragments retaining the hypervariable, antigen-binding region of an immunoglobulin and having a size similar to or smaller than a Fab′ fragment. This will include genetically engineered and/or recombinant proteins, whether single-chain or multiple-chain, which incorporate an antigen binding site and otherwise function in vivo as targeting vehicles in substantially the same way as natural immunoglobulin fragments. Preferred antibody fragments are single chain antibodies.

An Fv fragment is approximately 25,000 daltons and is the smallest fragment produced from IgG and IgM that contains a complete antigen binding site. Fv fragments have the same binding properties and similar three-dimensional binding characteristics as Fab (50,000 daltons). The V_{H} and V_{L} chains of the Fv fragments are held together by noncovalent interactions. These chains tend to dissociate upon dilution, so methods have been developed to crosslink the chains through glutaraldehyde, intermolecular disulfides or a peptide linker. Divalent single chain antibodies may also be produced by recombinant techniques.

Divalent single chain antibody fragments or subfragments, such as (sFv)₂ and (sFv′)₂, may be utilized in the methods of the present invention. The (sFv)₂ and (sFv′)₂ antibody fragments have two antigen binding sites and may provide better targeting and affinity to the antigen than monovalent antibodies with one antigen binding site, such as Fab′, Fab, Fv or Fv′. The fragments, (sFv)₂ and (sFv′)₂, are similar in molecular weight to the Fab′ (55,000 daltons) but are divalent rather than monovalent, thus providing an antibody fragment with better affinity and specificity to the lesion-associated antigen.

The tumor antigen recognized by the antibody fragments employed in the practice of the present invention may be one that is located on the cell surfaces of the tumor being targeted. A large number of solid tumor-associated antigens have now been described in the scientific literature, and the preparation and use of antibodies are well within the skill of the art. Of course, the tumor antigen that is ultimately selected will depend on the particular tumor to be targeted. Preferred tumor antigens are prostate specific membrane antigen (PSMA), carcinoembryonic antigen (CEA), CO17-1A and HER-2/neu. In a preferred embodiment, the antibody fragment is a single chain antibody.

In other embodiments, the TP is a tumor vasculature binding peptide. Targeting the tumor neovasculature with cytotoxic agents is advantageous for several reasons. Firstly, the target cells are directly accessible to intravenously administered therapeutic agents, permitting rapid localization of a high percentage of the injected dose (Kennel et al., 1991). Secondly, since each capillary provides oxygen and nutrients for thousands of cells in its surrounding ‘cord’ of tumor, even limited damage to the tumor vasculature could produce an avalanche of tumor cell death (Denekamp, 1990; Denekamp, 1984).

The tumor vasculature binding peptide can be any peptide that targets or homes to the tumor vasculature. Several peptide motifs that target the tumor vasculature have been identified. For example, by injecting phage peptide libraries into the circulation of nude mice bearing human breast carcinoma xenografts, Arap et al. identified three peptide motifs that home to tumors. (Arap et al., Science, 279:377-380, 1998). Additional tumor vasculature binding peptides can be identified using similar methods or other methods well known to those of skill in the art. Exemplary tumor vasculature binding peptides that can be used in the present invention are peptides comprising the Arg-Gly-Asp (RGD) motif, the Asn-Gly-Arg (NGR) motif or the Gly-Ser-Leu (GSL) motif.

The tumor vasculature binding peptide can be of any length. In preferred embodiments, the peptide comprises 3-15 amino acids. More preferably, the peptide comprises 11 amino acids. The peptide can be either in a linear or cyclic form. In preferred embodiments, the peptide is cyclic.

The Immune Response Triggering Portion (IRTP)

The IRTP may be any moiety that is capable of triggering an immune response. For example, the IRTP may be a moiety selected from the group consisting of an Fc fragment of immunoglobulin G (IgG), a fragment of the Fc fragment of IgG that exhibits the same biological function as the Fc region, or the extracellular domain of foreign major histocompatability complex (MHC).

The Fc fragment of immunoglobulin G (IgG) is responsible for immune responses induced by antibodies (Abbas, A. K., Lichtman, A. H., and Pober, J. S. 2000. Cellular and Molecular Immunology, W. B. Saunders Company, Philadelphia, Pa., pp. 60-61). First, Fc can trigger phagocytosis by neutrophils or macrophages because these cells express Fc receptors. Second, Fc is able to activate natural killer cells to lyse the cells bearing the Fc (Hu, Z., and Garen, A., 2000, Intratumoral injection of adenoviral vectors encoding tumor-targeted immunoconjugates for cancer immunotherapy, Proc. Natl. Acad. Sci. USA 97: 9221-9225). Third, it is the Fc portion of IgG that can trigger the complement system by binding with complement protein C1q. The Fc fragment of IgG has been utilized as a functional motif in tumor inhibition by fusing with other targeting proteins such as tissue factors (Hu, Z. and Garen, A., 2000, Intratumoral injection of adenoviral vectors encoding tumor-targeted immunoconjugates for cancer immunotherapy, Proc. Natl. Acad. Sci. USA 97: 9221-9225; Hu, Z., and Garen, A. 2001. Targeting tissue factor on tumor vascular endothelial cells and tumor cells for immunotherapy in mouse models of prostatic cancer, Proc. Natl. Acad. Sci. USA 98: 12180-12185).

The Fc fragment can be derived from any IgG. In some embodiments, the Fc fragment is derived from a mammalian IgG. Exemplary mammals from which the IgG may be derived are human, mouse, goat, cow and sheep. The IRTP can also be a fragment of the Fc fragment of IgG that exhibits the same biological function as the whole Fc fragment.

In other embodiments, the IRTP is the extracellular domain of foreign major histocompatibility complex (MHC). The extracellular domain of any MHC can be employed as the IRTP in the inventive antitumor agent. Foreign MHC has been shown to trigger an immune response. Tumorigenicity of tumor cells is significantly decreased when MHC molecules are introduced back into tumor cells by gene transfer (Tanaka, K., Isselbacher, K. J., Khoury, G., and Jay, G. 1985. Reversal of oncogenesis by the expression of a major histocompatibility complex class I gene. Science. 228:26-30; Nabel, G J., Yang, Z. Y., Nabel, E. G., Bishop, K., Marquet, M., Felgner, P. L., Gordon, D., and Chang, A. E. 1995. Direct gene transfer for treatment of human cancer. Ann. N. Y. Acad. Sci. 772:227-231), especially when foreign MHCs are transferred (O'neil, B. H., Kawakami, Y., Restifo, N. P., Bennink, J. R., Yewdell, J. W., and Rosenberg, S. A. 1993. Detection of shared MHC-restricted human melanoma antigens after vaccinia virus-mediated transduction of genes coding for HLA. J. Immunol. 151: 1410-1418; Nabel, E. G., Bishop, K., Marquet, M., Felgner, P. L., Gordon, D., and Chang, A. E. 1995. Direct gene transfer for treatment of human cancer. Ann. N. Y. Acad. Sci. 772:227-231.). The term “foreign” means that the MHC is derived from an organism other than the organism that is to be treated with the antitumor agent, or that the MHC is derived from an individual whose MHC does not match the individual that is to be treated with anitumor agent. Exemplary MHCs are H-2K^{d} and HLA-A0101.

In a preferred embodiment, the TP is a tumor vasculature binding peptide comprising the RGD motif (ACDCRGDCFCG), and the IRTP is the Fc fragment of mouse IgG at the C-terminus. The RGD peptide binds to αvβ3 integrins of the tumor vasculature and the Fc portion activates immune responses including the complement system, NK cells, neutrophils and/or macrophages to destroy the endothelial cells of blood vessels in tumors and tumor cells that express αvβ3 integrins.

Recent studies have demonstrated that not only new vasculature endothelial cells but also most of tumor cells express αvβ3 integrins (Pasqualini et al., Nature Biotech. 15:542-46 (1997)). Therefore, the RGD/mFc fusion protein should target both new blood vessels and existing tumor cells. As a result, this fusion protein may represent an agent that could eradicate tumors in addition to inhibiting tumor growth.

Synthesis Methods

The TP and the IRTP of the antitumor agent are linked to one another via bonding interactions. In some embodiments, the TP and the IRTP are linked via bonds formed between atoms in the peptide backbone of the TP and atoms in the peptide backbone of the IRTP. This type of linkage is referred to herein as “backbone-backbone” linkage. In other embodiments, the TP and the IRTP are linked via bonds formed between atoms in the peptide backbone of the TP and atoms in the amino acid side chains (surface) of the IRTP. The TP and the IRTP may also be linked via bonds formed between atoms in the amino acid side chains (surface) of the TP and atoms in peptide backbone of the IRTP. This type of linkage is referred to herein as “backbone-side chain” linkage. In some embodiments, the TP and the IRTP are directly linked to one another, while in other embodiments, there is a linking region between the TP and the IRTP.

The TP and IRTP may be linked in a backbone-backbone linkage via expression of a fusion protein by a host cell. In this embodiment, the antitumor agent is expressed in secreted form as a fusion protein of the TP and the IRTP. An expression vector comprising a nucleic acid sequence encoding the TP and the IRTP preceded by a nucleic acid sequence encoding a signal peptide is transfected into a host cell. The host cell expresses the fusion protein and the protein is purified using standard purification techniques. Methods of expression of a desired fusion protein by a host cell are well within the skill of the art.

The TP and IRTP may also be linked in a backbone-side chain linkage. Backbone-side chain linkage between the IRTP and the TP can be accomplished easily by those of ordinary skill in the art. Any method that attaches the IRTP and the TP via a backbone-side chain linkage is suitable for the present invention. Those of ordinary skill in the art are familiar with methods for achieving such coupling. The TP may be chemically modified so that each terminus of the backbone is a carboxylic acid group, using methods well known to those of skill in the art. The dicarboxylated TP can be coupled to the IRTP via surface amines of the IRTP. The amine found in the side chain of lysine may serve as a surface amine. The TP may also be chemically modified so that each terminus of the backbone is an amino group, using methods well known to those of skill in the art. The diamine derivatized TP can be coupled to the IRTP via surface carboxylic acids or carbohydrate moieties of the IRTP. The carboxylic acid found in the side chain of aspartate or glutamate residues can serve as a surface carboxylic acid. For example, the carbohydrate moiety of the Fc region of IgG may be coupled to a TP that has been derivatized to have an NH₂ group on its C-terminus.

Alternatively, the IRTP may be chemically modified so that each terminus of the backbone is a carboxylic acid group, using methods well known to those of skill in the art. The dicarboxylated IRTP can be coupled to the TP via surface amines of the TP. The IRTP may also be chemically modified so that each terminus of the backbone is an amino group, using methods well known to those of skill in the art. The diamine derivatized IRTP can be coupled to the TP via surface carboxylic acids or carbohydrate moieties of the TP.

An exemplary method for achieving such a coupling is EDC-mediated coupling of the C-terminal carboxylic acid of the TP to ethylene diamine, wherein EDC is 1-ethyl-3-(3-dimethylaminopropyl)carbodiimide hydrochloride. The derivatized TP is then coupled to the IRTP through its carbohydrate moiety via carbohydrate oxidation, Schiff's base formation and reduction. Many proteins contain carbohydrate moieties as a result of glycosylation. For example, a carbohydrate moiety is located in the Fc region of IgG.

In some embodiments, the IRTP and the TP are coupled via splicing methods. Exemplary splicing methods are described in Muir et al. Proc. Natl. Acad. Sci. USA 95 6705-6710 (1998) and Evans, Jr. et al. Protein Science 7 2256-2264 (1998), which are incorporated herein in their entirety.

In other embodiments, the linkage may be a “side-chain to side-chain” linkage. This linkage may be achieved by coupling a surface carboxylic acid on the IRTP with a surface amine on the TP or by coupling a surface amine on the TP with a surface carboxylic acid on the TP.

Pharmaceutical Compositions

Another aspect of the present invention is a pharmaceutical composition comprising the antitumor agent of the invention and a pharmaceutically suitable carrier or excipient. In some aspects of the present invention, the pharmaceutical composition comprises a vector that contains a DNA sequence encoding the antitumor agent of the invention and a pharmaceutically suitable carrier or excipient.

While the antitumor agent of the present invention or a vector containing a DNA sequence encoding the antitumor agent of the invention can be administered alone to a patient, the antitumor agent or vector of the present invention may also be present as part of a pharmaceutical formulation. Pharmaceutically suitable excipients typically include carriers known to those skilled in the art, including pharmaceutical adjuvants. Generally, these pharmaceutically acceptable carriers will include water, saline, buffers, and other compounds described, e.g., in the MERCK INDEX, Merck & Co., Rahway, N.J. See also Bioreversible Carriers in Drug Design, Theory and Application, Roche (ed.), Pergamon Press, (1987). These formulations typically comprise the pharmacological agent (i.e., the antitumor agent) in a therapeutically or pharmaceutically effective dose together with one or more pharmaceutically or therapeutically acceptable carriers and optionally other therapeutic ingredients. Various considerations are described, e.g., in Gilman et al. (eds.) (1990) Goodman and Gilman: The Pharmacological Bases of Therapeutics, 8th Ed., Pergamon Press; Novel Drug Delivery Systems, 2nd Ed., Norris (ed.) Marcel Dekker Inc. (1989), and Remington's Pharmaceutical Sciences, the full disclosures of which are incorporated herein by reference.

The compositions may be formulated in any pharmaceutical form appropriate for the desired route of administration. Examples of such compositions include solid compositions for oral administration such as tablets, capsules, pills, powders and granules which may be enteric coated or otherwise protected from hydrolysis, especially enzymatic hydrolysis, liquid compositions for oral administration such as solutions, suspensions, syrups or elixirs and preparations for parenteral administration such as sterile solutions, suspensions or emulsions. The compositions may also be manufactured in the form of sterile solid compositions which can be dissolved in sterile water, physiological saline or some other sterile injectable medium immediately before use.

Since the antitumor agent of the invention is amphoteric it may be utilized as free bases, as acid addition salts or as metal salts. The salts must be pharmaceutically acceptable, and these will include metal salts particularly alkali and alkaline earth metal salts, suitably potassium or sodium salts. A wide variety of pharmaceutically acceptable acid addition salts are available. These include those prepared from both organic and inorganic acids, preferably mineral acids. Typical acids which may be mentioned by way of example include citric, succinic, lactic, hydrochloric and hydrobromic acids. Such products are readily prepared by procedures well known to one skilled in the art.

In all such compositions, the antitumor agent or vector will normally be the principal physiologically active ingredient. The antitumor agent or vector may be formulated, however, with additional pharmacological agents for combination therapies. When used in treating cancer, for example, it may be formulated with compatible conventional chemotherapeutic agents.

Methods for administration are also discussed in Gilman et al. (eds.) (1990) and Norris (ed.), supra. In particular, the pharmaceutical composition of the invention may be administered intravenously, subcutaneously, orally, transdermally, such as in the method of (Prausnitz, M. R., Bose, V G, Langer, R, Weaver, J C: Electroporation of Mammalian skin: a mechanism to enhance transdermal drug delivery. Proc. Nat'l Acad. Sci. U.S.A., 90:10504-10508 (1993); and Wallace, B M, Lasker, J S: Stand and Deliver: getting peptide drugs into the body. Science 260:912-912 (1992). Liposomes may also be used to administer the procytotoxin of the invention (See Woodle, M C, Storm, G, Newman, M S, Jekot, J J, Collins, L R, Martin, F J, Szoka F C: Prolonged systemic delivery of peptide drugs by long circulating liposomes: illustration with vasopressin in the Brattleboro rat. Pharmaceut. Res. 9:260-265 (1992).

Optimal delivery routes, dosages and regimens for a given mammalian host can be readily ascertained by one skilled in the art. It will, of course, be appreciated that the actual dose used will vary according to the particular composition formulated, the particular compound used, the mode of application and the particular site, host and disease being treated. Many factors that modify the action of the drug will be taken into account including age, weight, sex, diet, time of administration, route of administration, rate of excretion, condition of the patient, drug combinations, reaction sensitivities and severity of the disease.

Methods of Treatment

The antitumor agent of the present invention is useful in various methods of treatment. The antitumor agent may be administered alone, or as a pharmaceutical composition described above. In some embodiments, the antitumor agent is employed in a method for inhibiting tumor growth. In other embodiments, the antitumor agent is used in a method for inhibiting angiogenesis. In some embodiments, the antitumor agent is employed in a method for treating a disease associated with neovascularization, such as cancer.

The antitumor agent is useful for treating a variety of cancers, such as primary or metastatic solid tumors, including lung, colorectal, bladder, prostate, breast, renal, brain, pancreatic, melanoma, head and neck and ovarian cancer.

The methods can be conducted in vitro or in vivo. Exemplary in vitro methods may involve contacting a cell with an effective amount of the antitumor agent. Exemplary in vivo methods involve administering to a patient in need thereof an effective amount of the antitumor agent. As used herein, the term “patient” includes both humans and other species; thus the invention has both medical and veterinary applications.

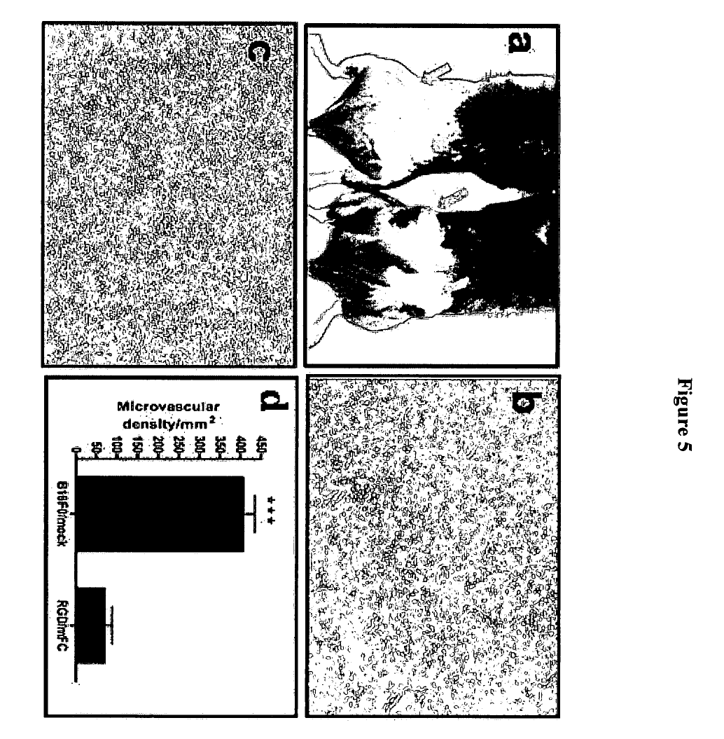

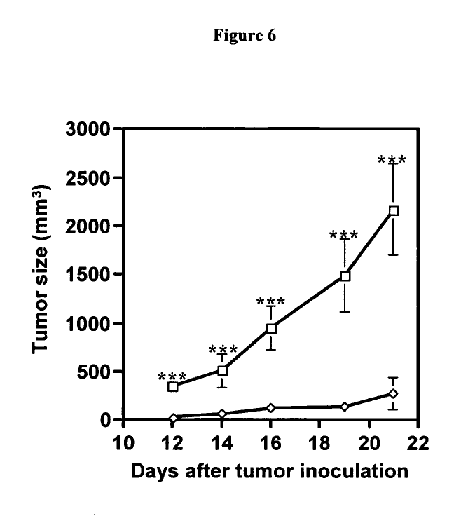

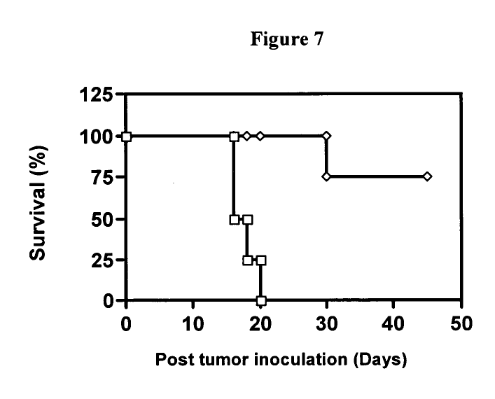

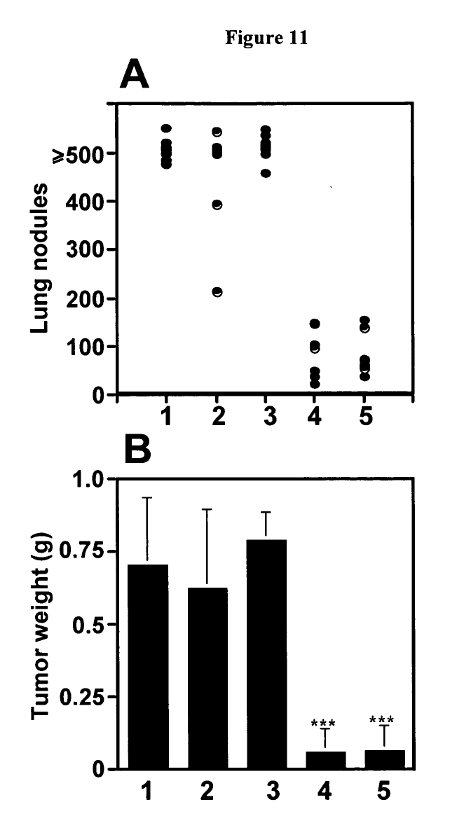

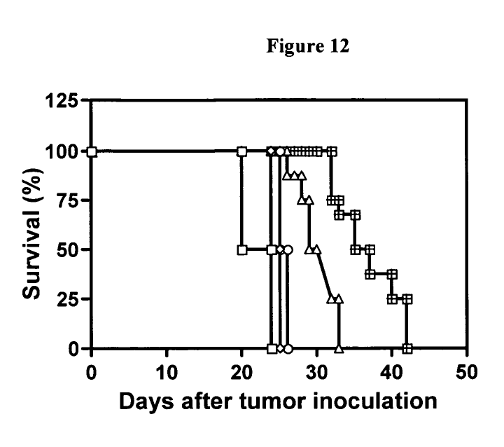

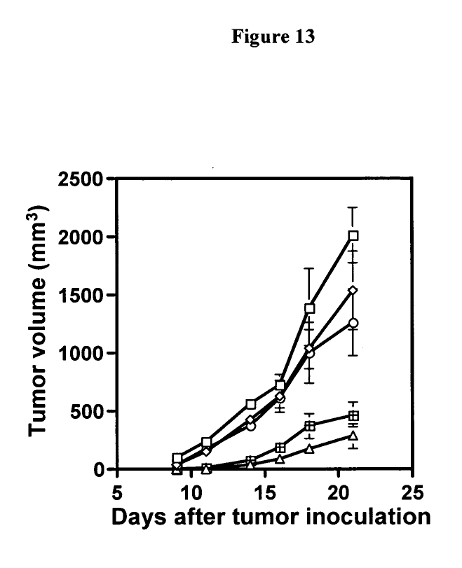

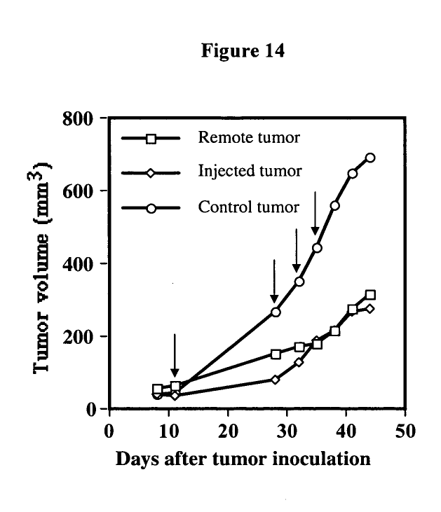

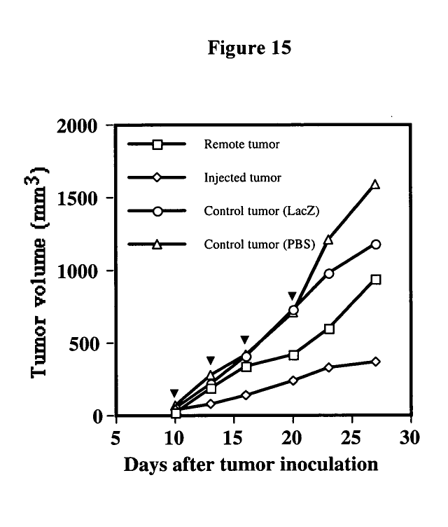

These methods of treatment are exemplified in the following Examples. For example, the tumors of mice that were injected with B16F0 cells expressing the RGD/mFc fusion protein had dramatically fewer new blood vessels and were smaller than the tumors of mice that were injected with B16F0 cells. See FIGS. 5 and 6. Additionally, mice that were injected with B16F0 cells expressing the RGD/mFc fusion protein had a better survival rate than mice that were injected with B16F0 cells. See FIG. 7. Mice that were injected intravenously with B16F0/PSMA5/H2K^{d}/scPSMA cells generated significantly fewer tumor nodules as compared to the control group. See FIG. 11A. The size of the tumors generated in mice that were injected with B16F0/PSMA5/H2K^{d}/scPSMA cells were smaller than the tumors of control mice. See FIG. 11B. A survival study showed that the fusion protein H2K^{d}/scPSMA significantly increased overall survival. See FIG. 12. Similar results were obtained when mice were injected intradermally. See FIG. 13. Additionally, the tumors of mice that were injected with adenovirus particles comprising an adenovirus vector containing RGD/mFc fusion gene were smaller than the tumors of mice that were injected with adenovirus particles comprising an adenovirus vector containing LacZ gene. See FIGS. 14 and 15.

### EXAMPLES

Materials and Methods for Examples 1-3

Animals. Female C57BL/6J mice, 6-8 week of age, were purchased from Jackson Laboratories (Bar Harbor, Me.) and housed in a pathogen-free animal facilities.

Cell lines. B16F0 mouse melanoma cells (ATCC #CRL 6322, Rockville, Md.) were cultured in DMEM (GIBCO BRL, Grand Island, N.Y.) containing 10% fetal bovine serum (FBS, Hyclone, Logan, Utah) and 50 μg/ml of gentamicin (GIBCO BRL). As used herein, the term “regular B16F0 cells” means B16F0 cells that have not been altered in any way (i.e., these cells have not been altered to express RGD/mFc or to already be expressing PMSA).

Western Blot And FACS Analysis. The supematants of B16F0 cells and RGD/mFc expressing B16F0 were run on SDS-PAGE and transferred onto nitrocellular film. The fusion protein was recognized by an anti-Fc fragment antibody (Bethyl Laboratories, Montgomery, Tex.) and the Super Signal West Pico Chemiluminescent detection kit (Pierce, Rockford, Ill.). Stable cell lines that express RGD/mFc were also analyzed by intracellular Fluorescence Activated Cell Sorting (FACS). Briefly, the secretion of the protein was blocked and the cells were permeabilized with a Cytofix/Cytoperm Plus Kit (BD PharMingen, San Diego, Calif.). The cells were then stained with FITC conjugated anti-mouse Fc antibody and analyzed using the FACS Calibur (Becton/Dickinson, San Jose, Calif.).

Matrigel Plug. 5×10⁵ B16F0 cells genetically engineered to express RGD/mFc protein were mixed with 0.2 ml of liquid Matrigel (Becton/Dickinson) and injected subcutaneously into female C57BL/6J mice. Two control experiments were also completed. In one control experiment, 0.2 ml of liquid Matrigel Matrigel was injected subcutaneously into female C57BL/6J mice. In the second control experiment, 5×10⁵ B16F0 cells were mixed with 0.2 ml of liquid Matrigel and injected subcutaneously into female C57BL/6J mice. At day 8, the mice were intravenously injected with 50 μg/mouse of FITC Lectin (Sigma, St. Luis, Mich.). Thirty minutes later, the Matrigel implants were isolated. After a quick observation under a fluorescent microscope, they were frozen for sectioning. Thick sections (20 μm) were made and observed using fluorescent microscope.

Immunohistochemical Analysis. 5×10⁵ RGD/mFc expressing B16F0 cells in 0.2 ml of PBS were subcutaneously injected into C57BL/6J mice. As a control experiment, 5×10⁵ B16F0 cells in 0.2 ml of PBS were subcutaneously injected into C57BL/6J mice. Fourteen days later, the tumors were excised and frozen sections were made. Slides were made using these frozen sections. The slides were then fixed and immnuohistochemically stained with anti-CD31 antibody and visualized with a Vectastain Elite ABC kit (Vector Laboratories, Burlingame, Calif.).

The above described Matrigel implants were also studied by immunohistochemical analysis. Slides were made with the frozen sections. The slides were then fixed and immnuohistochemically stained with anti-CD31 antibody and visualized with a Vectastain Elite ABC kit (Vector Laboratories, Burlingame, Calif.).

Animal Study. 2×10⁵ RGD/mFc expressing B16F0 cells in 0.2 ml of PBS were subcutaneously injected into C57BL/6J mice. As a control experiment, 2×10⁵ B16F0 cells in 0.2 ml of PBS were subcutaneously injected into C57BL/6J mice. Tumors were measured with a digital caliper and statistically analyzed and recorded. Tumor volume was calculated as ½ length×width². For the survival study, when tumors reached the size of 20 mm in diameter, the mouse carrying it was considered dead. Each group had 8 mice. Student T test was used to analyze the tumor volume data.

### Example 1

### Construction of a RGD/mFc Fusion Protein

Construction Of The RGD/Mfc Fusion Protein Vector And Transfection Into B16F0 Tumor Cells. PCR was used to amplify the Fc fragment (from the hinge region to the end of the cDNA, 741 bp) of mouse IgG cDNA sequence (ATCC #MGC-6628). A pair of primers (shown below) was designed to be used in a PCR reaction. At the N-terminus of the 5′ primer, after an enzyme cutting site (HindIII), a sequence encoding ACDCRGDCFCG peptide was added. A BamHI enzyme site was added to the N-terminus of the 3′ primer.

5′ primer:
CCAAGCTTGCCTGCGACTGCCGAGGAGATTGTTTCTGCGGCGACAAGAAA
ATTGTGCCCAGGGATTGTGGTTGTAAGCCTTGC
3′ primer:
CGGGATCCCGTTTACCAGGAGAGTGGGAGAG

At the 5′ end of the up-stream primer, after a Hind III enzyme site, a sequence encoding ACDCRGDCFCG peptide was added. A BamHI enzyme site was added to the 5′ end of the down-stream primer.

The PCR product was then inserted into the expression vector pSecTag2B (containing a useful histidine tag), in frame, after the signal peptide sequence. The full sequence was confirmed by sequencing. The generated vector, pRGD/mFc, was then transfected into B16F0 tumor cells using Lipofectamine transfection reagents according to the manufacturer's protocol (Gibco BRL). Stable cell lines were selected from DMEM containing 200 or 500 μg/ml Zeocin (Sigma, St. Louis, Mo.) for 3-4 weeks.

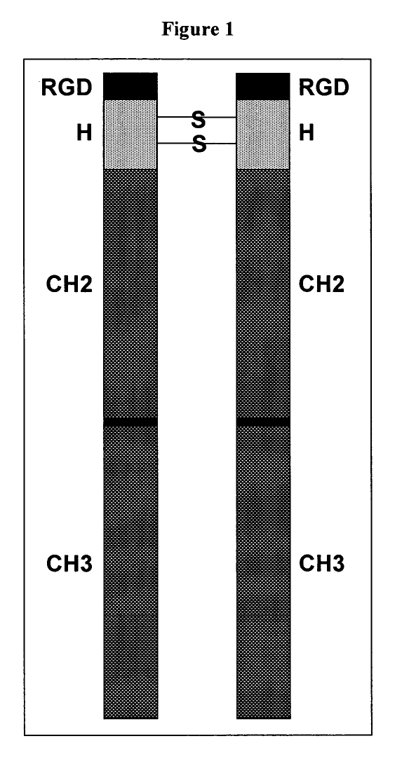

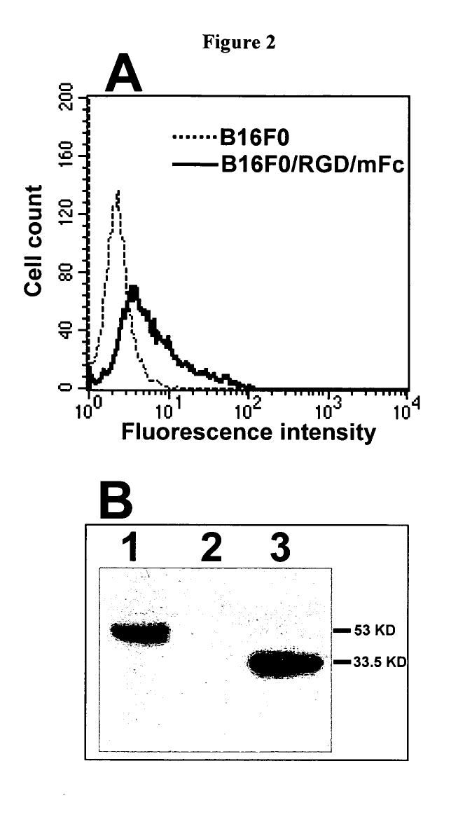

RGD/mFc expression. FIG. 1 shows a diagram of the fusion protein, RGD/mFc. It contains a RGD peptide sequence (ACDCRGDCFCG) in the front followed by the hinge region, CH2, and CH3 regions of mouse IgG. To confirm the expression of the fusion protein, the vector pRGD/mFc was linerarized and transfected into B16F0 melanoma cells and stable expressing cell lines were obtained. Intracellular FACS analysis using anti-mouse Fc fragment antibody demonstrated that the protein is expressed within these cells (FIG. 2A) and Western blot analysis showed that the protein is secreted into the culture medium (FIG. 2B).

### Example 2

### Inhibition of Tumor Angiogenesis Caused by RGD/mFc Fusion Protein

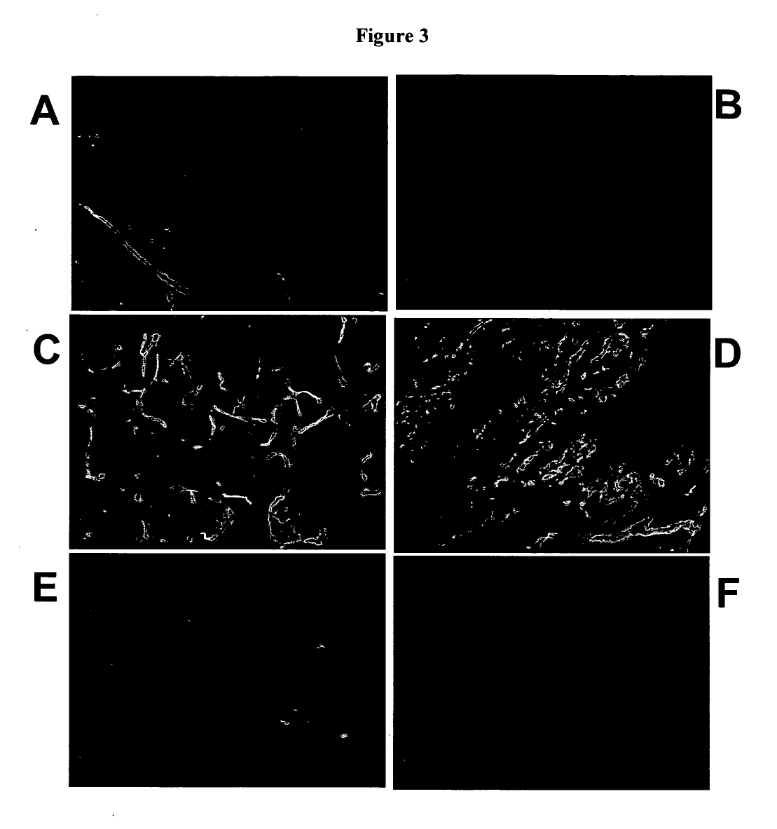

The sections of the Matrigel implants were observed using fluorescent microscope. For the first control experiment (Matrigel only injected subcutaneously into mice), some blood vessels were observed on the surface of the Matrigel implant, as shown in FIG. 3A. No vessels were observed inside the implant. For the second control experiment (B16F0 cells and Matrigel injected subcutaneously into mice) B16F0 tumor cells dramatically enhanced new blood vessel formation in the Matrigel implant, as shown in FIGS. 3C and 3D. In contrast, for the experiment in which B16F0 cells genetically engineered to express RGD/mFc protein and Matrigel were injected subcutaneously into mice, the formation of new blood vessels in the Matrigel implant was dramatically decreased, as shown in FIGS. 3E and 3F, indicating that the RGD/Fc fusion protein has anti-angiogenesis activity.

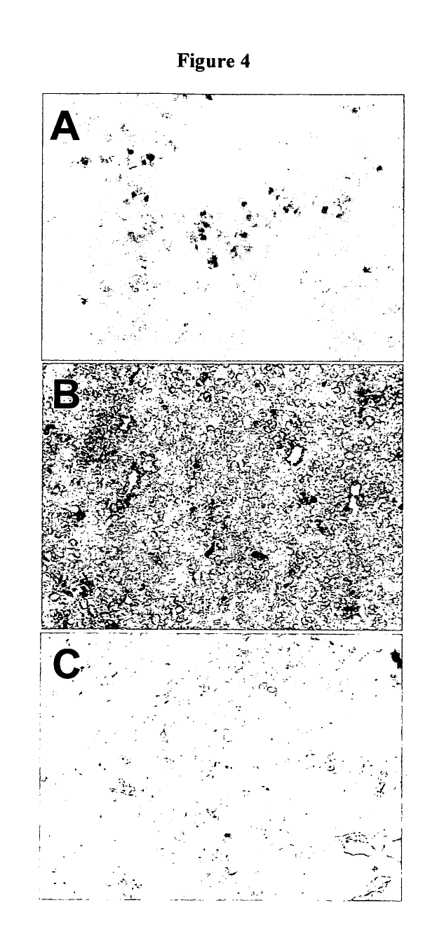

Immunohistochemical analysis of the sections from the Matrigel implants showed that not only are there dramatically fewer blood vessels but also the tumor cell mass is much less in the Matrigel implant containing RGD/mFc expressing B16F0 cells compared to the Matrigel implant containing regular B16F0 cells (See FIG. 4). In another tumor model, TC-1, similar anti-angiogenesis effects were observed (data not shown).

To further confirm this observation, tumor cells (either B16F0 cells in PBS or B16F0 expressing RGD/mFc in PBS) were subcutaneously injected into mice. Fourteen days later, the tumors were excised and frozen section slides were made and immnuohistochemically stained with anti-CD31 antibody. Once again, dramatically fewer new blood vessels were observed in the tumors from RGD/mFc expressing B16F0 cells compared to the tumors from regular B16F0 cells (See FIGS. 5B and 5C). It is noted that there is a significant difference in size between tumors of B16F0 cells and B16F0 cells expressing RGD/mFc (See FIG. 5A). The apparent difference in tumor cell number between RGD/mFc expressing tumors in the Matrigel implant and those without Matrigel in FIGS. 4C and 5C is an artifact of the different cell densities in these two tumor models.

### Experiment 3

### Inhibition of Tumor Growth Caused by RGD/mFc Fusion Protein

To statistically determine whether the RGD/mFc fusion protein inhibits tumor growth, B16F0 tumor cells expressing RGD/mFc protein were subcutaneously injected into female C57BL/6J mice. As a control experiment, B16F0 tumors cells were injected into separated groups of mice. The tumor volumes were measured, statistically analyzed, and plotted. As shown in FIG. 6, a significant size difference between tumors from mice in the control experiment and tumors from mice injected with RGD/mFc expressing B16F0 cells was observed. The tumors from mice in the control experiment were larger than the tumors from mice injected with RGD/mFc expressing B16F0 cells. For example, at day 21, a statistically significant difference in tumor size was observed (p<0.001).

The survival of the mice injected with RGD/mFc expressing B16F0 cells was also monitored. As shown in FIG. 7, the RGD/mFc fusion protein significantly prolonged survival time of the mice. When the tumor size reaches 20 mm in diameter, the mouse is considered dead. At day 45, 75% of the mice bearing cells expressing RGD/mFc were still alive while all the mice in the control group (mice that were injected with unaltered B16F0 cells) died before day 20 (FIG. 7).

Materials and Methods for Examples 4-5

### Example 4

### Construction and Transfection of pH2-K^{d}/PMSA_{VH+VL} and Expression of H2K^{d}/scPSMA

Total RNA were isolated from lymphocytes of BALB/c mice and the H2-K^{d} cDNA was generated by RT-PCR using primers: 5′-CAGTGCGATGGCACCCTGCACGC-3′ and 5′-GGCAGCTGTCTTCACGCTAGAGAATG-3′, and inserted into vector pCR2.1 (pH2-K^{d}). The cDNA sequence encoding the heavy chain of anti-human PSMA was cloned from total RNAs isolated from 7E11 hybridoma cell line (ATCC #HB10494) by reverse transcription using a 3′ primer in the constant region:

5′-GACACTGGGATCATTTACCAGGAGAG-3′

and 5′-RACE using primers:

5′-CGACTGGAGCACGAGGACACTGA-3′
and
5′-GACACTGGGATCATTTACCAGGAGAG-3′.

The cDNA was then inserted into vector pCR4-TOPO (pPSMA_{H}). Similarly, the cDNA sequence encoding the light chain of anti-human PSMA was cloned by reverse transcription using oligo dT primer and amplification using primers:

5′-CGACUGGAGCACGAGGACACUGA-3′ and
5′-GAGCTGGTGGTGGCGTCTCAG-3′. (pPSMA_{L})

The extracellular domain of H-2K^{d} cDNA was amplified from pH2-K^{d} by PCR using primers:

5′-GGAATTCATGGCACCCTGCACGCTGC-3′ and
5′ GCTTACGTTAACCACAATCCCTGGGCAGGCGCG
CCCGAGACAGTGGATGGAGGAAG-3′.

To keep the hinge region at the 5′ end of the down stream primer, the Hpa I restriction enzyme site and half of the hinge region sequences were added. Similarly, the variable region of anti-human PSMA heavy chain was amplified from pPSMA_{H} by PCR using primers:

5′ ATTGTGGTTAACGTAAGCCTTGCATATGTACAGGCCG
TACGACCA AGAGCACACCTGCAAATG-3′ and
5′ CCGCTCGAGGCTCTAGATTACCAGGTGCTGGAGGGGA
CAGTCAC-3′.

The Hpa I restriction enzyme site and the other half of the hinge region sequences were added at the 5′ end of the up stream primer. The two PCR products were then digested with Hpa I/EcoR I and Hpa I/Xho I, respectively, ligated, and cloned into pCR2.1 (pH2-K^{d}/PSMA_{H}). Using a similar strategy, the variable region of the light chain of anti-human PSMA was added into pH2-K^{d}/PSMA by two separated PCR and PCR fragment ligation. The primers are:

5′-GGAATTCATGGCACCCTGCACGCTGC-3′ and
5′ GCCCCCCCGGGGGGCCCACCAGAAGCAGGGCCTCCGCTCGCC
CAGGTGCTGGAGGGGACAGTC-3′
for pH2-K^{d}/PSMA_{H}, and
5′ GCCCCCCGGGGGGGCCTCCGGTGGCCCGGCCAGTGGGGGACC
CGCATCAGGAGGCCCTGATGTTGTGATGACCCAGACTC-3′ and
5′-CCGCTCGAGTCACCGTTTTATCTCCAGCTTGGTCCCCCC-3′
for pPSMA_{L}.

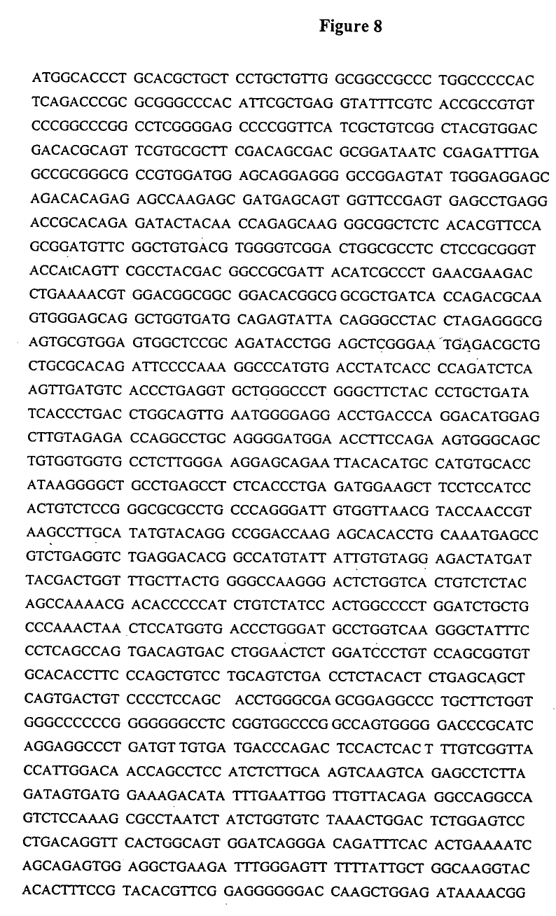

A linker sequence was introduced between the variable region of the heavy chain and the variable region of the light chain. The final plasmid, pH2-K^{d}/PSMA_{VH+VL}, contains the following components: the extracellular domain of H-2K^{d}, the hinge region, the variable region of anti-PSMA heavy chain, a linker sequence, and the variable region of anti-PSMA light chain, as shown in FIG. 8.

The pH2-K^{d}/PSMA_{VH+VL} was then transfected into B16F0 tumor cells and B16F0/PSMA5 cells, respectively, using Lipofectamine transfection reagents according to the manufacturer's protocol (Gibco BRL). Stable cell lines were selected using G418 resistance and maintained in culture.

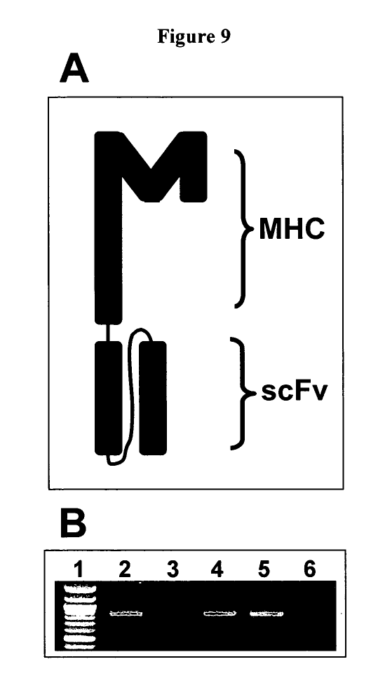

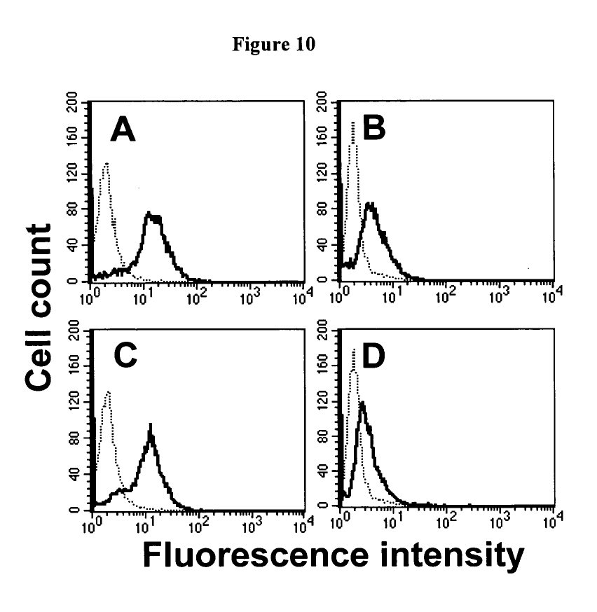

H2K^{d}/scPSMA expression. FIG. 9A shows a diagram of the H2K^{d}/scPSMA fusion protein. Plasmid pH2-K^{d}/PSMA_{VH+VL} was transfected into B16F0 tumor cells and stable cell clones were selected. RT-PCR was performed to detect the transcripts of the fusion gene. In the four stable clones analyzed, three were RT-PCR positive (See FIG. 9B). The fusion protein expression was further confirmed by intracellular FACS analysis among the RT-PCR positive clones using antibodies against mouse IgG and H-2K^{d}, respectively. FIG. 10 shows two exemplary clones chosen by FACS analysis. Both of the clones expressed high levels of the extracellular domain of H-2K^{d} (FIGS. 10A and 10C). Signals were also detected for the V_{H}V_{L} portion of the fusion protein (FIGS. 10B and 10D).

### Example 5

### Inhibition of Tumor Growth and Improvement of Overall Survival Caused by H2K^{d}/scPSMA

B16F0/PSMA5 cells were transfected with pH2-K^{d}/PSMA_{VH+VL}. Several clones were obtained and the H2K^{d}/scPSMA expression was confirmed by RT-PCR and intracellular FACS analysis. Two of the clones, B16F0/PSMA5/H2K^{d}/scPSMA#3 and B16F0/PSMA5/H2K^{d}/scPSMA #7, along with other control cells such as B16F0, B16F0/PSMA5, and B16F0/H2K^{d}/scPSMA were used in the following animal tumor studies.

A pulmonary metastasis assay was performed by injecting 5×10⁵ cells intravenously into female C57BL/6J mice via the tail vein. Three weeks later, the mice were sacrificed and the tumor nodules on the lungs were counted. B16F0/PSMA5/H2K^{d}/scPSMA cells (clone #3 and #7) generated a significantly lower number of tumor nodules compared to the control groups (FIG. 11A). The size of the tumor nodules generated from B16F0/PSMA5/H2K^{d}/scPSMA cells were dramatically smaller than those nodules generated from the control cells. This was monitored by total tumor weight measurements and is presented in FIG. 11B. A survival study showed that the fusion protein H2K^{d}/scPSMA significantly increased overall survival (P<0.01, FIG. 12).

An intradermal tumor study was performed by injecting 2×10⁵ tumor cells in 0.2 ml PBS on the right flank of female C57BL/6J mice (8 mice/group). After day 8, the tumors were measured every other day. Tumor volume was calculated as ½ length×width². B16F0/PSMA5/H2K^{d}/scPSMA tumor cells grew significantly slower than the control cells. (B16F0/PSMA5/H2K^{d}/scPSMA#3 vs. B16F0, p<0.02;
B16F0/PSMA5/H2K^{d}/scPSMA#3 vs. B16F0/PSMA, p<0.002;
B16F0/PSMA5/H2K^{d}/scPSMA#3 vs. B16F0/H2K^{d}/scPSMA, p<0.02;
B16F0/PSMA5/H2K^{d}/scPSMA#7 vs. B16F0, p<0.03; B16F0/PSMA5/H2K^{d}/scPSMA#7 vs. B16F0/PSMA, p<0.003; B16F0/PSMA5/H2K^{d}/scPSMA#7 vs. B16F0/H2K^{d}/scPSMA, p<0.05) This was especially true during the early stage of the tumor development (FIG. 13). At days 11 and 14, the p values between B16F0/PSMA5/H2K^{d}/scPSMA#3 and the control cells, and between B16F0/PSMA5/H2K^{d}/scPSMA#7 and the control cells are all smaller than 0.001.
   Materials and Methods for Examples 6-7

Animals. Female C57BL/6J mice, 6-8 weeks of age, were purchased from Jackson Laboratories (Bar Harbor, Me.) and housed in a pathogen-free animal facility.

Cell lines. Murine tumor cells, TC-1, were cultured in RPMI 1640 medium containing 10% FBS, 0.2 mM non-essential amino acids, 2 mM L-glutamine, 1 mM sodium pyruvate, and 50 μg/ml gentamicin at 37° C. with 5% CO₂. HEK293 cells were cultured in EMEM medium containing 10% heat-inactivated horse serum, 1 mM sodium pyruvate, 0.1 mM non-essential amino acids, 1.5 g/L sodium bicarbonate, and 50 μg/ml gentamicin at 37° C. with 5% CO₂. Human prostate tumor cells, DU-145 were cultured in EMEM medium containing 10% FBS, 1 mM sodium pyruvate, 0.1 mM non-essential amino acids, 1.5 g/L sodium bicarbonate, and 50 μg/ml gentamicin at 37° C. with 5% CO₂.

Construction of Adenovirus Vector Containing RGD/mFc Fusion Gene. The fusion gene (RGD/mFc) was first cloned into a shuttle vector. The shuttle vector containing the fusion gene was then inserted into an adenoviral vector (pAdEasy-1) by homologous recombination to form the recombinant Ad plasmid. After linearization, the plasmid was transfected into HEK293 cells, which contain the necessary genes for adenovirus to replicate. Virus particles were harvested from the cells and tittered.

Construction of Adenovirus Vector Containing LacZ Gene. The LacZ gene was first cloned into a shuttle vector. The shuttle vector containing the LacZ gene was then inserted into an adenoviral vector (pAdEasy-1) by homologous recombination to form the recombinant Ad plasmid. After linearization, the plasmid was transfected into HEK293 cells, which contain the necessary genes for adenovirus to replicate. Virus particles were harvested from the cells and tittered.

### Example 6

### Inhibition of Prostate Tumor Growth by RGD/mFc

Human prostate tumor cells (DU-145) were subcutaneously injected into nude mice (5×10⁶ cells per site) at both left and right flanks. Eleven, twenty-eight, thirty-two and thirty-five days after the initial injection of DU-145 cells, virus particles comprising an adenovirus vector containing RGD/mFc fusion gene were directly injected into the tumors (1×10⁸ PFU/tumor in 50 μl solution). As controls, adenovirus particles comprising an adenovirus vector containing LacZ gene were injected in the same doses on the same days after the initial injection of DU-145 cells. Measurements of tumor size were taken 8, 11, 28, 32, 35, 38, 41, and 44 days after the initial injection of DU-145 cells. The tumor size was calculated as ½ length×width² (See FIG. 14).

The size of the tumor injected with virus particles comprising an adenovirus vector containing RGD/mFc fusion gene was smaller than the size of the tumor injected with adenovirus particles comprising an adenovirus vector containing LacZ gene. Additionally, the size of a remote tumor that was not injected with virus particles comprising an adenovirus vector containing RGD/mFc fusion gene was smaller than the size of the tumor injected with adenovirus particles comprising an adenovirus vector containing LacZ gene.

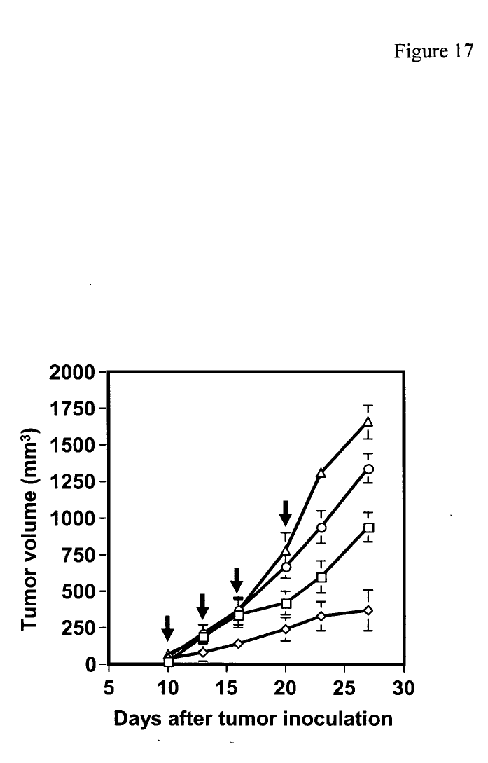

Thus, as shown in FIG. 17, RGD/mFc effectively inhibited DU-145 growth in nude mice when compared to controls, indicating that the anti-tumor effect was achieved by either the complement system or natural killer cells or both. Surprisingly, the inhibition effect of RGD/mFc on distant tumors is almost as effective as the injected tumor in the nude mice model; while in the TC-1 tumor model, the distant tumors were inhibited but not as effective as the injected tumor. The anti-angiogenesis effect of RGD/mFc may vary in different types of cells, especially when the tumor cells are from different species. It is possible that they may express the target molecules, αvβ3 at different levels and the in vivo transduction efficiency may be different as well.

### Example 7

### Inhibition of TC-1 Tumor Cell Growth by RGD/mFc

TC-1 murine tumor cells were subcutaneously injected into C57BL/6J mice (1×10⁵ cells per site) at both the left and right flanks. Ten, thirteen, sixteen and twenty days after the initial injection of TC-1 cells, virus particles comprising an adenovirus vector containing RGD/mFc fusion gene were directly injected into the tumors (1×10⁸ PFU/tumor in 50 μl solution). As controls, adenovirus particles comprising an adenovirus vector containing LacZ gene were injected in the same doses on the same days after the initial injection of TC-1 cells. Measurements of tumor size were taken 10, 13, 16, 20, 23, and 27 days after the initial injection of TC-1 cells. The tumor size was calculated as ½ length×width² (See FIG. 15).

The size of the tumor injected with adenovirus particles comprising an adenovirus vector containing RGD/mFc fusion gene was smaller than the size of the tumor injected with adenovirus particles comprising an adenovirus vector containing LacZ gene. Additionally, the size of a remote tumor that was not injected with virus particles comprising an adenovirus vector containing RGD/mFc fusion gene was smaller than the size of the tumor injected with adenovirus particles comprising an adenovirus vector containing LacZ gene.

### Example 8

### Synthesis of RGD/mFc Antitumor Agent (“Backbone-side Chain” Linkage)

The protein sequence ACDCRGDCFCG is derivatized to contain an amine at both the N and C terminal end by coupling a diamine to the C terminal carboxylic acid group. The peptide is dissolved in 0.1 M MES, 0.5 M NaCl, pH 6.0 to form solution A. The mFc is dissolved in 0.1 M MES, 0.5 M NaCl, pH 6.0 at a concentration of 1 mg/ml to form solution B. To 1 ml of solution B, 0.4 mg EDC (˜2 mM) and 0.6 mg of NHS is added. The solution is allowed to react for 15 minutes at room temperature. 1.4 μl of 2-mercaptoethanol (final concentration of 20 mM) is added to quench the EDC. Solution A is added in an amount to provide an equal mole-to-mole ratio of ACDCRGDCFCG and mFc. The reaction continues at room temperature for 2 hours. The reaction is quenched by adding hydroxylamine to a final concentration of 10 mM. The reaction is purified by gel filtration.

### Experiment 9

### Treatment of a Human Patient with Metastatic Lung Cancer

A 175-pound male human patient diagnosed with metastatic lung cancer could be given therapy with RGD/mFc antitumor agent. The patient may be infused intravenously with a therapeutically effective amount of antitumor agent, such as 50 mg RGD/mFc, and the treatment may be repeated weekly for a suitable period of time following this initial treatment, such as 4 weeks. A suitable amount of time after the final dose of RGD/mFc, such as four months, a computerized tomography scan of the patient would ideally show no evidence of lung cancer, and all signs and symptoms of the disease would not be evident.

### Example 10

### RGD/mFc Inhibition of Tumor Metastases

In order to determine if RGD/mFC can inhibit tumor metastases, 5×10⁵ B16F0/mock or RGD/mFC expressing B16F0 cells were intravenously injected into female C57BL/6J mice through tail vein. As controls, B16F0/mock cells were also injected in the same manner. Lung metastases measured as lung nodules 24 days after the tumor inoculation are presented as Table 1. Both the RGD/mFc expressing clones generated a significantly less lung tumor nodules (9.625±4.09 and 23.875±7.98, respectively) than the control tumor cells (>200).

TABLE 1
Pulmonary metastatic assay

| Tumor cells | Lung tumor nodules |
|---|---|
| B16F0/mock | >200, >200, >200, >200, >200, 186, >200, >200 |
| B16F0/mFc #15 | 4, 5, 12, 14, 13, 6, 8, 15 |
| B16F0/mFc #17 | 8, 28, 32, 17, 26, 20, 26, 34 |

Different tumor cell lines were i.v. injected into C57BL/6J mice (5×10⁵/mouse). Twenty-four days later, the mice were sacrificed and the tumor nodules on the lungs were counted.

### Example 11

### Adenovirus Vector Construction to Express RGD/mFc

A recombinant adenoviral vector containing the coding sequence for the RGD/mFc fusion protein was constructed by using the AdEasy Adenoviral Vector System (Stratagene, La Jolla, Calif.). Briefly, the RGD/mFc sequence was amplified from the pRGD/mFc and cloned into the pShuttle-CMV vector; the resulting pShuttle-CMV/RGD/mFc was then transformed into BJ5183-AD-1 electorporation competent cells to generate recombinant adenoviral vector pAd-RGD/mFc. Following Pac I digestion to linearize the pAd-RGD/mFc viral vector, it was transfected into AD-293 cells by using the Transfection MBS Mammalian Transfection Kit (Stratagene). Ten days later, primary viral stocks were prepared from the transfected cells and viral titer determined with BD Biosciences' Adeno-X Rapid Titer Kit. Amplification of recombinant virus was performed by infecting 125×10⁶ AD-293 cells at a MOI of 3 for about four days until cytopathic effects were complete. Finally, the recombinant virus was harvested and purified by using BD Biosciences' Adeno-X Virus Purification Kits according to the instructions provided. The purified virus was then titered, aliquoted, and stored at −80° C. for future use. Production of RGD/mFc fusion protein was confirmed by Western Blot from different cells (AD-293 cells, TC-1 cells, etc) infected with pAd-RGD/mFc. AD-LacZ from Stratagene was amplified and titered as stated above and used as the control.

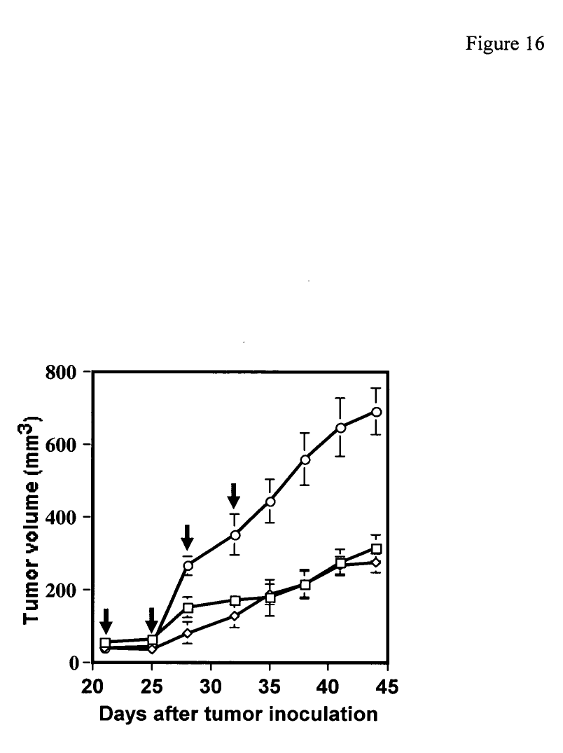

After directly injecting 1×10⁸ PFU of the virus into tumors (TC-1 tumor in C57BL/6J mice and DU-145 tumor in nude mice), a significant delay of tumor growth was observed in both tumor models (FIGS. 16 and 17). For TC-1 tumor cells, a significant difference between the virus injected tumors and control tumors (AD-LacZ injected tumors or PBS injected tumors) was observed three days after the first virus injection (P=0.017 and P=0.016). For DU-145 tumor cells, a significant difference between the virus injected tumors and AD-LacZ injected tumors was observed three days after the second virus injection (P=0.023). Furthermore, the growth of the tumors in the left flanks (uninjected) of the mice was significantly inhibited as well, compared to that of the controls.

To test RGD/mFc's anti-tumor growth effect as an agent, an adenovirus vector encoding RGD/mFc fusion protein was constructed and transfected into AD-293 cells, from which adenovirus particles were isolated. These replication deficient virus particles were directly injected into tumors. The results demonstrated that RGD/mFc expressed by the virus-infected cells inhibits tumor growth in both murine and human tumors. More importantly, the expressed fusion protein enters the circulation system and reaches distant tumors and inhibits their growth (FIG. 7) with no obvious adverse effects. It is impossible to deliver genes or proteins directly into all tumors in humans; therefore, newly developed anti-tumor agents must be delivered systemically and be able to reach distant tumors.

1. An antitumor agent comprising a targeting portion and an immune response triggering portion, wherein
(a) said targeting portion is selected from the group consisting of an antibody fragment and a tumor vasculature binding peptide; and
(b) said immune response triggering portion is selected from the group consisting of an Fc fragment of immunoglobulin G (IgG), a fragment of the Fc fragment of IgG that exhibits the same biological function as the Fc region, and the extracellular domain of foreign major histocompatability complex (MHC).

2. The antitumor agent of claim 1, wherein said antibody fragment is a single chain antibody.

3. The antitumor agent of claim 2, wherein said antibody fragment binds to a tumor antigen selected from the group consisting of prostate specific membrane antigen (PSMA), CEA, CO17-1A and HER-2/neu.

4. The antitumor agent of claim 1, wherein said tumor vasculature binding peptide comprises arginine-glycine-aspartate (RGD), asparagine-glycine-arginine(NGR), or glycine-serine-leucine (GSL).

5. The antitumor agent of claim 1, wherein said IgG is from a mammal.

6. The antitumor agent of claim 5, wherein said mammal is a selected from the group consisting of human, mouse, goat, cow and sheep.

7. The antitumor agent of claim 6, wherein said mammal is a human.

8. The antitumor agent of claim 1, wherein said foreign MHC is selected from the group consisting of H-2Kd, and HLA-A0101.

9. The antitumor agent of claim 1, further comprising a pharmaceutically acceptable carrier.

10. The antitumor agent of claim 1, wherein said targeting portion and said immune response triggering portion are fused via backbone-backbone linkage.

11. The antitumor agent of claim 1, wherein said targeting portion and said immune response triggering portion are fused via backbone-side chain linkage.

12. An expression vector comprising a nucleotide sequence encoding the antitumor agent of claim 10.

13. A method for inhibiting tumor growth comprising administering to a patient in need thereof an effective amount of the antitumor agent of claim 1.

14. A method for inhibiting tumor angiogenesis comprising administering to a patient in need thereof an effective amount of the antitumor agent of claim 1.

15. A method for treating a disease associated with neovascularization, comprising administering to a patient in need thereof, an effective amount of the antitumor agent of claim 1.

16. The method of claim 15, wherein said disease is cancer.


SEQUENCE LISTING (19 sequences)

SEQ ID NO: 1 - DNA, 1749 bp, Artificial Sequence
  Description of Artificial Sequence Synthetic polynucleotide construct
atggcaccctgcacgctgctcctgctgttggcggccgccctggcccccactcagacccgcgcgggcccacattcgctgaggtatttcgtcaccgccgtgtcccggcccggcctcggggagccccggttcatcgctgtcggctacgtggacgacacgcagttcgtgcgcttcgacagcgacgcggataatccgagatttgagccgcgggcgccgtggatggagcaggaggggccggagtattgggaggagcagacacagagagccaagagcgatgagcagtggttccgagtgagcctgaggaccgcacagagatactacaaccagagcaagggcggctctcacacgttccagcggatgttcggctgtgacgtggggtcggactggcgcctcctccgcgggtaccatcagttcgcctacgacggccgcgattacatcgccctgaacgaagacctgaaaacgtggacggcggcggacacggcggcgctgatcaccagacgcaagtgggagcaggctggtgatgcagagtattacagggcctacctagagggcgagtgcgtggagtggctccgcagatacctggagctcgggaatgagacgctgctgcgcacagattccccaaaggcccatgtgacctatcaccccagatctcaagttgatgtcaccctgaggtgctgggccctgggcttctaccctgctgatatcaccctgacctggcagttgaatggggaggacctgacccaggacatggagcttgtagagaccaggcctgcaggggatggaaccttccagaagtgggcagctgtggtggtgcctcttgggaaggagcagaattacacatgccatgtgcaccataaggggctgcctgagcctctcaccctgagatggaagcttcctccatccactgtctccgggcgcgcctgcccagggattgtggttaacgtaccaaccgtaagccttgcatatgtacaggccggaccaagagcacacctgcaaatgagccgtctgaggtctgaggacacggccatgtattattgtgtaggagactatgattacgactggtttgcttactggggccaagggactctggtcactgtctctacagccaaaacgacacccccatctgtctatccactggcccctggatctgctgcccaaactaactccatggtgaccctgggatgcctggtcaagggctatttccctcagccagtgacagtgacctggaactctggatccctgtccagcggtgtgcacaccttcccagctgtcctgcagtctgacctctacactctgagcagctcagtgactgtcccctccagcacctgggcgagcggaggccctgcttctggtgggccccccgggggggcctccggtggcccggccagtgggggacccgcatcaggaggccctgatgttgtgatgacccagactccactcactttgtcggttaccattggacaaccagcctccatctcttgcaagtcaagtcagagcctcttagatagtgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaagatttgggagttttttattgctggcaaggtacacactttccgtacacgttcggaggggggaccaagctggagataaaacgg

SEQ ID NO: 2 - protein, 11 aa, Artificial Sequence
  Description of Artificial Sequence Synthetic peptide
ACDCRGDCFCG

SEQ ID NO: 3 - DNA, 83 bp, Artificial Sequence
  Description of Artificial Sequence Primer
ccaagcttgcctgcgactgccgaggagattgtttctgcggcgacaagaaaattgtgcccagggattgtggttgtaagccttgc

SEQ ID NO: 4 - DNA, 31 bp, Artificial Sequence
  Description of Artificial Sequence Primer
cgggatcccgtttaccaggagagtgggagag

SEQ ID NO: 5 - DNA, 23 bp, Artificial Sequence
  Description of Artificial Sequence Primer
cagtgcgatggcaccctgcacgc

SEQ ID NO: 6 - DNA, 26 bp, Artificial Sequence
  Description of Artificial Sequence Primer
ggcagctgtcttcacgctagagaatg

SEQ ID NO: 7 - DNA, 26 bp, Artificial Sequence
  Description of Artificial Sequence Primer
gacactgggatcatttaccaggagag

SEQ ID NO: 8 - DNA, 23 bp, Artificial Sequence
  Description of Artificial Sequence Primer
cgactggagcacgaggacactga

SEQ ID NO: 9 - DNA, 26 bp, Artificial Sequence
  Description of Artificial Sequence Primer
gacactgggatcatttaccaggagag

SEQ ID NO: 10 - RNA, 23 nt, Artificial Sequence
  Description of Artificial Sequence Primer
cgacuggagcacgaggacacuga

SEQ ID NO: 11 - DNA, 21 bp, Artificial Sequence
  Description of Artificial Sequence Primer
gagctggtggtggcgtctcag

SEQ ID NO: 12 - DNA, 26 bp, Artificial Sequence
  Description of Artificial Sequence Primer
ggaattcatggcaccctgcacgctgc

SEQ ID NO: 13 - DNA, 56 bp, Artificial Sequence
  Description of Artificial Sequence Primer
gcttacgttaaccacaatccctgggcaggcgcgcccgagacagtggatggaggaag

SEQ ID NO: 14 - DNA, 63 bp, Artificial Sequence
  Description of Artificial Sequence Primer
attgtggttaacgtaagccttgcatatgtacaggccgtacgaccaagagcacacctgcaaatg

SEQ ID NO: 15 - DNA, 44 bp, Artificial Sequence
  Description of Artificial Sequence Primer
ccgctcgaggctctagattaccaggtgctggaggggacagtcac

SEQ ID NO: 16 - DNA, 26 bp, Artificial Sequence
  Description of Artificial Sequence Primer
ggaattcatggcaccctgcacgctgc

SEQ ID NO: 17 - DNA, 63 bp, Artificial Sequence
  Description of Artificial Sequence Primer
gcccccccggggggcccaccagaagcagggcctccgctcgcccaggtgctggaggggacagtc

SEQ ID NO: 18 - DNA, 80 bp, Artificial Sequence
  Description of Artificial Sequence Primer
gccccccgggggggcctccggtggcccggccagtgggggacccgcatcaggaggccctgatgttgtgatgacccagactc

SEQ ID NO: 19 - DNA, 39 bp, Artificial Sequence
  Description of Artificial Sequence Primer
ccgctcgagtcaccgttttatctccagcttggtcccccc